(19) United States
(12) Patent Application Publication
(10) Pub. No.: US 2008/0306457 A1
(43) Pub. Date: Dec. 11, 2008

(54) Enhanced surgical related liquid collection

(75) Inventor: Julie A. Ryan, Hoover, AL (US)

Correspondence Address: WILLIAM W. HAEFLIGER, 201 S. LAKE AVE, SUITE 512, PASADENA, CA, 91101, US

(73) Assignee: BASS Medical, Inc.

(21) Appl. No.: 11/810,040

(22) Filed: Jun. 5, 2007

(51) Int. Cl. A61B 19/00

(52) U.S. Cl. 604/317

(57) ABSTRACT

Liquid collection apparatus usable during surgery, comprising in combination, first and second collectors arrayed and configured for receiving liquid dropping from a surgery area or areas, the collectors arrayed in selected edgewise proximal relation to enhance surgical related liquid collection and transfer capability.

### BACKGROUND OF THE INVENTION

This invention relates generally to liquid collection apparatus usable during surgery to collect liquid drainage from the surgery area; and more particularly concerns an easily manipulated liquid collection tray and liquid collection means in the tray allowing surgeon treading over the collector.

During surgery, liquid such as sterile water supplied or used as irrigation liquid is commonly allowed to drain to the floor and collects there. This increases floor slipperiness and interferes with surgical procedures. There is need for improvements in such collection and disposal methods, as well as ease of handling and manipulation of collection and disposal apparatus, as well as ease of rolling travel of operating room heavy equipment over such apparatus, and need for foot comfort of persons standing in such a surgery area.

### SUMMARY OF THE INVENTION

It is a major object of the invention to provide improved apparatus meeting the above needs. Basically, the improved liquid collection apparatus usable during surgery comprises

a) first and second collectors arrayed and configured for receiving liquid dropping from a surgery area or areas,

b) the collectors arrayed in selected edgewise proximal relation to enhance surgical related liquid collection capability.

Another object includes provision of attachment structure for attaching the collectors in the arrayed relation, whereby the second collector provides a selected position extension of the first collector.

A further object includes provision of porous means facing upwardly, at each collector for receiving dropping liquid and to allow treading of a surgeon or other surgical personnel, during surgery.

An added object includes provision of collectors selectively connected to provide upward facing areal continuity.

Yet another object includes provision of a second collector having an alternate folded position to extend over a first collector, for draining liquid from the second collector onto the first collector, for subsequent removal from the first collector.

Yet further object includes provision of:
i) collectors mutually arrayed in L-shaped (winged) configuration;
ii) collectors arrayed in end-to-end configuration;
iii) collectors spaced apart to receive liquid drainage from surgery areas which are spaced apart relative to a surgery table;
iv) collectors having interfitting portions proximate edges of the collectors;
v) tubing at one of the collectors for receiving and emptying fluid from the one collector;
vi) tubing extending between the collectors for receiving and emptying fluid from one collector into the other collector, the tubing for example being ported for fluid inflow;
vii) at least one collector comprising a flexible impermeable tray adapted for flex-folding over the other collector;
viii) porous material in one collector extends into edge adjacency to porous material in the other collector;
ix) connection of the adjacent collectors by tape, or other means as will be seen.

These and other objects and advantages of the invention, as well as the details of an illustrative embodiment, will be more fully understood from the following specification and drawings, in which:

### DRAWING DESCRIPTION

FIG. 1 shows two collectors in edge proximity, one collector having fluid removal tubing;

FIG. 2 shows two collectors extending in edge proximity, to form a wing shape, proximate a surgical area or zone;

FIG. 3 is a side view showing two collectors, one folded or displaced to extend over the other, for fluid drainage from one to the other, for removal;

FIG. 4 is an enlarged vertical section to show interior construction of edge-adjacent collectors, hinge connected; and FIG. 4a is a vertical section showing a modification;

FIG. 5 is a plan view of small and large collectors adjacent a surgery area;

FIG. 6 is a plan view of two collectors with fluid drainage tubing extending from one to the other;

FIG. 7 is an elevation view of ported drainage tubing;

FIG. 8 is a section showing a modification of edge-to-edge connection of two collectors;

FIG. 8a is a section showing a further modification; and

FIGS. 9-11 are sections showing various porous matrix edge-to-edge configurations.

### DETAILED DESCRIPTION

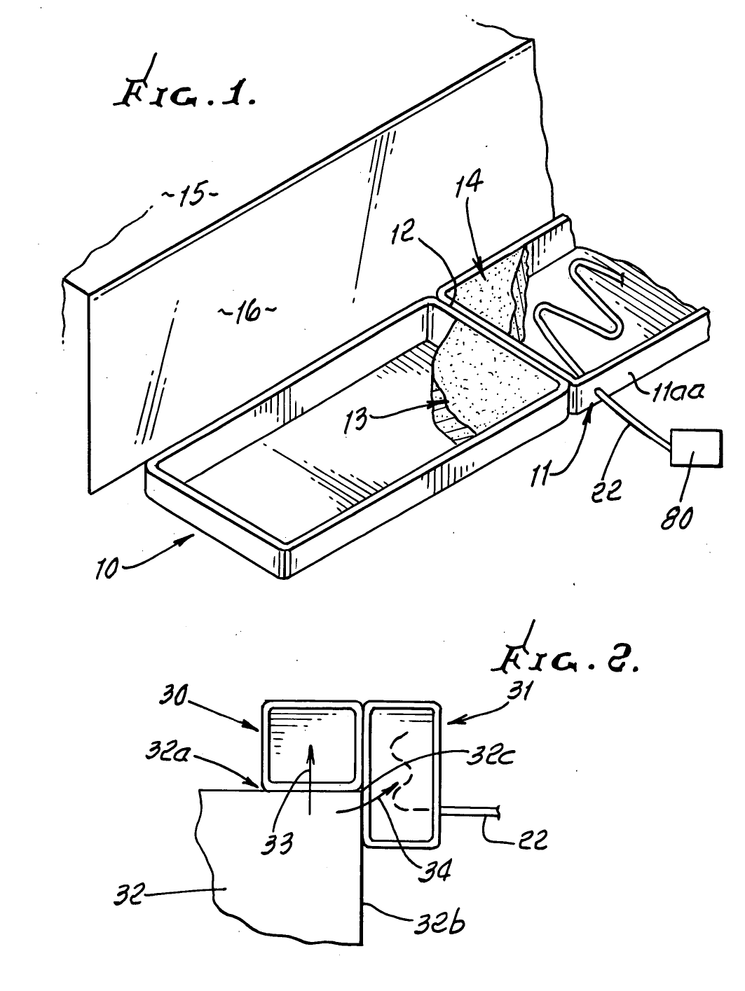

FIG. 1 shows first and second collectors 10 and 11 in edge-to-edge adjacent relation at zone 12. The collectors may comprise shallow plastic trays containing porous matrix material such as batting sheets 13 and 14 placed into the trays to enable treading of surgical personnel on the sheets. Liquid drains off the operating table 15 or off sheets 16, into the trays. Each tray may consist of lightweight flexible plastic foam, such as closed cell polyethylene, enabling easy folding of each tray, and the sheets 13 and 14. FIG. 4 shows edge-to-edge location of the two trays 10 and 11, with tray upstanding walls 10a and 11a meeting at interface 12. Liquid falling on tops of the walls drains into the two trays (see arrows 17 and 17′). The walls may be interconnected as by flexible tape 18 on tops of the walls as shown, and the walls may also, or alternatively, be interconnected by flexible tape 19 under the walls, as shown in FIG. 4a. This allows relative parallel shifting of the walls 10a and 11a to selected positions.

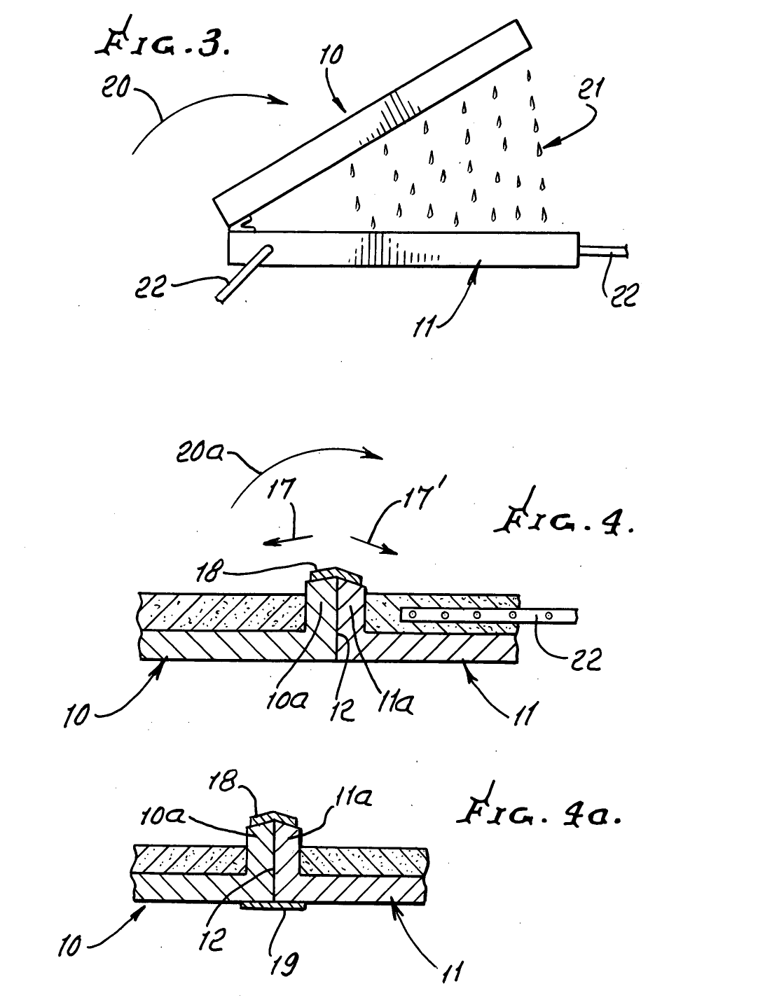

If only tape 18 is used, the collector 10 can be easily displaced upwardly and also guidedly inverted (tape 18 acting as a hinge), as by swinging it as indicated by arrows 20 in FIGS. 3, and 20a in FIG. 4, to drain liquid from collector 10, at 21, into collector 11, for removal, as via a ported tube 22. That tube can be deployed with undulation as shown in FIG. 1, and it exits the tray 11, via edge wall 11aa, to extend to a suction device, such as a pump 80 for liquid removal from tray 11. Thus, tray 10 serves as an auxiliary tray, needing no liquid removal tubing, but still allowing liquid removal, as referred to.

FIG. 2 shows two trays 30 and 31, like trays 10 and 11, respectively, adjunct tray 30 being the smaller and extending at right angular wing configuration relative to tray 31, as at one end 32a of operating table 32. Tray 31 extends on the floor at the lower side 32b of table 32. The arrangement is such that the corner 32c of the table is bounded as shown by the two trays, liquid from the table flowing at 33 and 34 into the trays.

FIG. 8 shows a modification allowing edge-to-edge meeting at 40 of the porous matrix material layers 41 and 42 in two trays 43 and 44, that otherwise correspond generally to trays 10 and 11 referred to. A substantially continuous upper surface 44 is thereby provided by the porous matrix layers, for liquid collection. Note that matrix material layer 41 overlaps the edge 43a of its tray 43, and that tray edge 44a of tray 44 underlaps the edge 42a of matrix material layer 42, whereby joint 45 between 43 and 44 is offset relative to the joint 46 between 41 and 42. Joint 46 is angled, as shown, to facilitate upward folding inversion of tray 43 (see arrow 99) to allow liquid drainage from tray 43 into tray 44 associated with suction tubing 47. FIG. 8a shows the two trays being moved relatively together, for assembly. If only tray 44 is used, tape 49 may be extended adjacent the edge of matrix material 42, to serve as a barrier to side drainage.

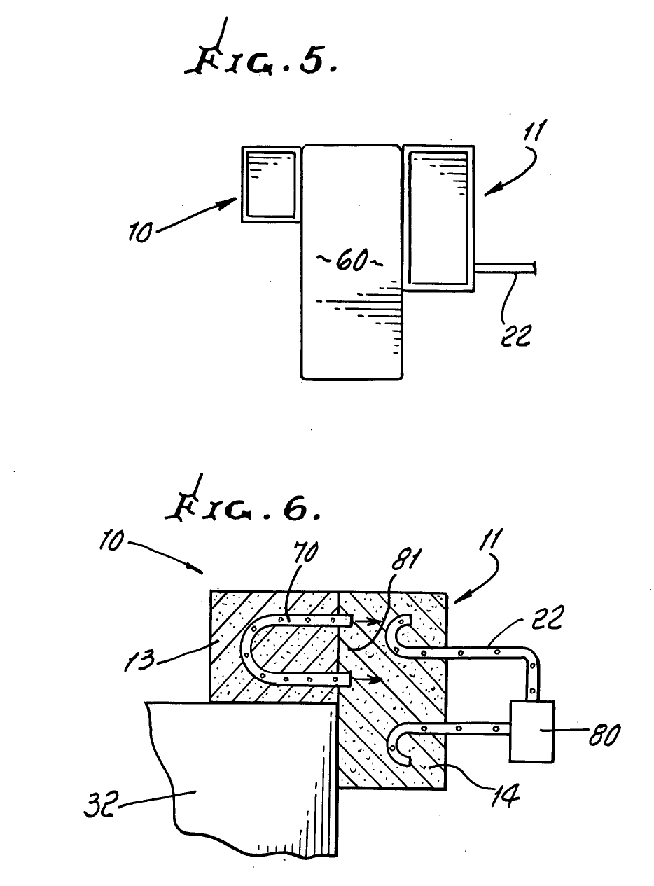

FIG. 5 shows use of two trays, such as 10 and 11, separated, and located at opposite sides of operating table 60. Periodically, tray 10 can be emptied into tray 11, from which liquid is removed by tubing 22.

FIG. 6 shows drainage tubing 70 extending from adjacent the matrix material 13 in tray 10 into the matrix material 14 in edge-adjacent tray 11, for drainage into that tray in response to suction applied via tubing 22 in tray 11. See suction pump 80 connected to tubing 22. The latter extends into proximity to tubing 70, as at 81, to assist in draining liquid from tubing 70 into tray 11.

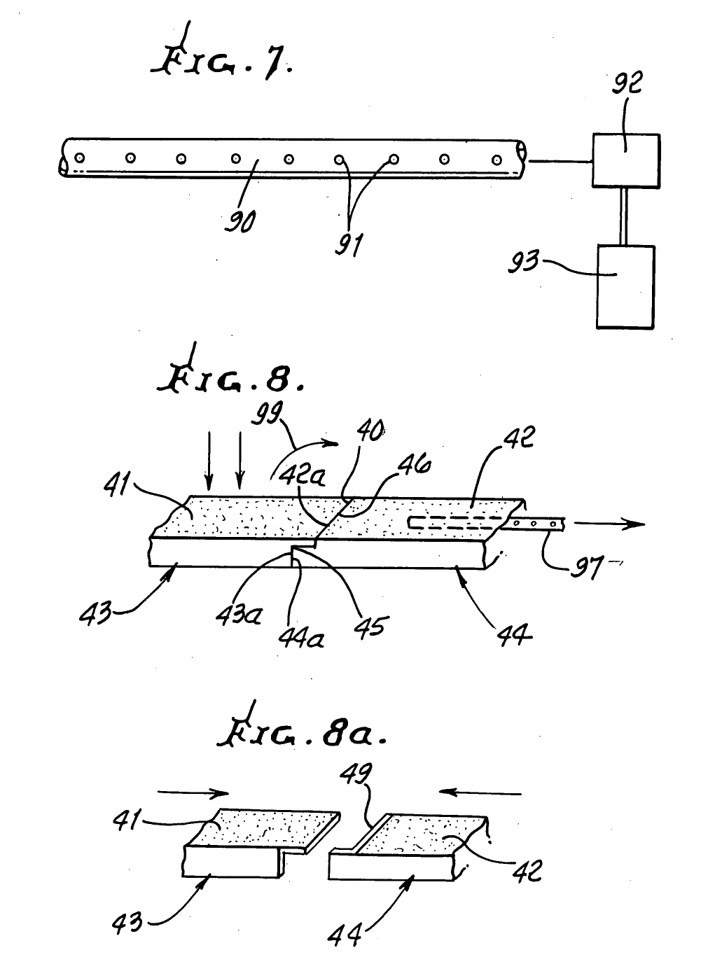

FIG. 7 shows typical suction tubing 90, having side intake parts 91, for liquid, along its length. Also shown is a suction pump 92, discharging to a collection canister 93, for disposal.

The collectors, and porous matrix material are typically flexible, and easily foldable, for rapid and sanitary disposal.

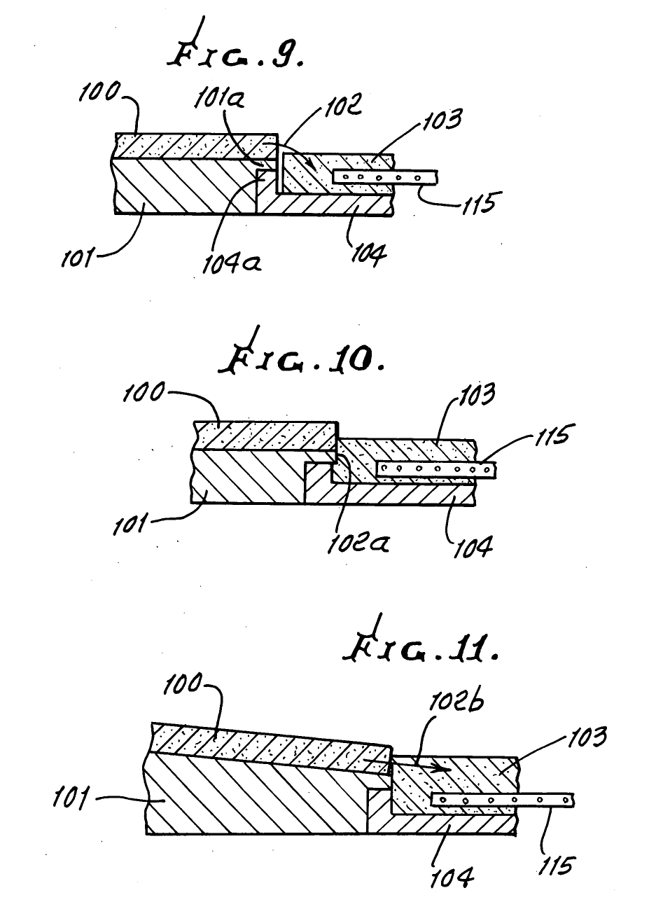

FIGS. 9-11 show three additional modifications. In FIG. 9, porous matrix 100 from collector 101 extends into edge-to-edge proximity to porous matrix 103 in collector 104 that has liquid removal tubing 115. Matrix 100 extends over ledge 101a of 101, and over upright sidewall 104a of 104. Liquid from 100 drains into matrix 103, as indicated by arrow 102.

The same elements are shown in FIG. 10, except that matrix 100 engages matrix 103, at edge-to-edge contact locus 102a, and liquid from 100 drains into 103 since 100 is at higher level than 103.

In FIG. 11, the matrix 100 is tilted downwardly toward matrix 103 and toward engagement locus at 102b enhancing drainage of liquid from 100 into 103. In FIGS. 9-11, the matrix 100 in collector 101 has direct edge-to-edge exposure to matrix 103 in collector 104.

I claim:

1. Liquid collection apparatus usable during surgery, comprising in combination:
a) first and second collectors arrayed and configured for receiving liquid dropping from a surgery area or areas,
b) said collectors arrayed in selected edgewise proximal relation to enhance surgical related liquid collection and transfer capability.

2. The combination of claim 1 including attachment structure for attaching said collectors in said arrayed relation, whereby the second collector provides a selected position extension of the first collector.

3. The combination of claim 1 including porous means facing upwardly, at each collector for receiving said dropping liquid and to receive treading of a surgeon or other surgical personnel during surgery, whereby liquid collection into the porous means and transfer therefrom is enhanced.

4. The combination of claim 1 wherein said collectors are selectively edge connected to provide upward facing areal continuity, in different collector edgewise parallel shifted positions.

5. The combination of claim 1 wherein the second collector has an alternate folded position, to extend over the first collector for draining liquid from the second collector onto the first collector, for subsequent removal from the first collector.

6. The combination of claim 2 wherein said structure comprises tape extending protectively over adjacent edges of both collectors.

7. The combination of claim 2 wherein said structure comprises tape extending under adjacent edges of both collectors.

8. The combination of claim 1 wherein said collectors are mutually arrayed in L-shaped configuration.

9. The method of providing for collection of liquid draining off a table or the like, as during surgery, that includes:
a) providing first and second collectors arrayed and configured for receiving liquid dropping from a surgery area or areas,
b) said collectors arrayed in selected edgewise proximal relation to enhance surgical related liquid collection capability from both collectors as via the second collector.

10. The method of claim 9 including connecting said collectors to remain in said proximal relation, despite treading thereon, as during surgery.

11. The method of claim 10 wherein said connecting includes applying tape to extend protectively over adjacent parallel edges of both collectors.

12. The method of claim 10 including applying tape to extend under adjacent edges of both collectors.

13. The method of claim 9 wherein said collectors are arrayed in L-shaped configuration.

14. The method of claim 9 including displacing the second collector to extend over the first collector for draining liquid onto the first collector to be suction removed.

15. The method of claim 14 wherein said displacing includes at least partially inverting the second collector to assist said draining.

16. Liquid collection apparatus usable during surgery together with a first collector that receives liquid dropping from a surgery area, or areas, comprising
a) a second liquid collector configured to be arrayed for receiving liquid dropping from a surgery area or areas,
b) said second collector having an elongated edge configured to be extended in proximal and parallel relation to an elongated edge of the first collector, to receive liquid dropping from a surgical area or areas, thereby to enhance surgical related liquid collection capability of both said first and second collectors operating together.

17. The apparatus of claim 16 wherein the collectors together extend in L-shaped array configuration.

18. The apparatus of claim 16 including means associated with the second collector for securing the second collector adjacent the first collector.

19. The apparatus of claim 16 wherein the second collector extends over and above the first collector for draining collected liquid onto the first collector.

20. The apparatus of claim 1 including first and second porous matrices in the respective collectors, said matrices extending into edge-to-edge proximal relation.

21. The apparatus of claim 20 wherein the first matrix extends at a higher level than the second matrix proximate edges thereof.

22. The apparatus of claim 20 wherein the first matrix engages the second matrix, at edges thereof.

23. The apparatus of claim 20 wherein the first matrix is tilted down and toward the second matrix proximate edges thereof.
